(19) United States
(12) Patent Application Publication
(10) Pub. No.: US 2020/0003795 A1
(43) Pub. Date: Jan. 2, 2020

(54) DETERMINATION OF ANALYTES IN LIQUID SAMPLES BY MASS SPECTROMETRY

(71) Applicant: Quest Diagnostics Investments Incorporated, Wilmington, DE (US)

(72) Inventors: Sum CHAN, San Clemente, CA (US); Richard E. REITZ, Las Vegas, NV (US); Mildred M. GOLDMAN, Laguna Niguel, CA (US); Seyed A. SADJADI, Laguna Niguel, CA (US); Michael P. CAUFIELD, Oceanside, CA (US); Darren A. CARNS, San Juan Capistrano, CA (US)

(21) Appl. No.: 16/567,360

(22) Filed: Sep. 11, 2019

Related U.S. Application Data
(63) Continuation of application No. 13/102,993, filed on May 6, 2011, now abandoned.
Application No. 13/102,993 is a division of application No. 10/290,104, filed on Nov. 5, 2002, now abandoned.
(60) Provisional application No. 60/333,091, filed on Nov. 5, 2001.
(60) Provisional application No. 60/332,529, filed on Nov. 5, 2001.
(60) Provisional application No. 60/333,089, filed on Nov. 5, 2001.
(60) Provisional application No. 60/333,090, filed on Nov. 5, 2001.

(51) Int. Cl. G01N 33/92

(52) U.S. Cl. CPC G01N 33/92

(57) ABSTRACT

The present invention relates to compositions and methods for analyzing analytes of interest in liquid samples by mass spectrometry, and preferably in patient samples. Preferred analytes of interest include sirolimus (rapamycin), corticosteroids, bile acids and lamotrigine (lamictal). In one embodiment, by careful selection of target ions, a number of corticosteroids can be analyzed simultaneously and without interference from closely related molecules. In another embodiment, the present methods combine high turbulence liquid chromatography with mass spectrometry performed in positive and negative mode in a single assay to enable the detection and quantification of the composit˜on of bile acid pools. By combining mass spectrometry and high-throughput chromatography, the methods and compositions described herein can provide a rapid, sensitive, and accurate assay for use in large clinical laboratories.

### RELATED APPLICATIONS

This application is a divisional application of U.S. application Ser. No. 13/102,993, filed on May 6, 2011, which is a divisional application of U.S. application Ser. No. 10/290,104, filed on Nov. 5, 2002, which claims the benefit of U.S. Provisional Application Ser. Nos. 60/333,091; 60/332,529; 60/333,090; and 60/333,089; each of which was filed on Nov. 5, 2001, and the contents of each of which are incorporated by reference herein in their entirety, including all tables, figures, and claims.

### FIELD OF THE INVENTION

The present invention relates generally to compositions and methods for analyzing analytes of interest in liquid samples by mass spectrometry, and preferably in patient samples. In particular, the methods and compositions described herein provide rapid, sensitive, and accurate assays for sirolimus (rapamycin), corticosteroids, bile acids and lamotrigine (lamictal), and are particularly applicable to the large clinical laboratory.

### BACKGROUND OF THE INVENTION

The following discussion of the background of the invention is merely provided to aid the reader in understanding the invention and is not admitted to describe or constitute prior art to the present invention.

Assays for Sirolimus

Sirolimus, also known by the name rapamycin and the trade name Rapamune® (Wyeth-Ayerst), is a macrocyclic compound produced by Streptomyces hygroscopicus that exhibits immunosuppressive properties in various animals, including humans. Sirolimus inhibits T-cell activation and proliferation by binding to FK Binding Protein 12 (FKBP-12), resulting in creation of an immunosuppressive complex that inhibits progression through the cell cycle. This mechanism is distinct from that of other well known immunosuppressive agents such as cyclosporine.

Sirolimus is rapidly absorbed on administration, with a mean-time-to-peak concentration of 1-2 hours. The mean blood-to-plasma ratio of sirolimus is 36±17.9, indicating that the compound is substantially sequestered in blood components. Sirolimus is metabolized by cytochrome P450 IIIA4 and P-glycoprotein, with seven major metabolites detectable in blood, including hydroxy-, demethyl-, and hydroxydemethyl-forms of the compound. In addition to blood, serum, and plasma, sirolimus is also detectable in fecal and urine samples. Therapeutic levels of sirolimus in blood are about 3-18 ng/mL (trough).

Mass spectrometry, particularly tandem mass spectrometry, in combination with off-line extraction and/or chromatographic preparation of samples prior to injection into the mass spectrometer, has been applied to the measurement of sirolimus in clinical specimens. Such assays are disclosed in, e.g., Taylor et al., Ther. Drug Monitoring 22:608-12 (2000); Salm et al., Clin. Therapeutics 22, Supl. B:B71-B85 (2000); and Hallensleben et al., J. Am. Soc. Mass Spectrom. 11:516-25 (2000).

Assays for Corticosteroids

Corticosteroids are steroid derivatives that are produced by the adrenal cortex in both humans and non-human animals. Their production is under the control of pituitary hormones, and they are intimately involved in energy metabolism in the body. Adult humans secrete about 20 mg of cortisol and about 2 mg of corticosterone per day, with greater amounts being produced in times of stress. The amount of cortisol present in the body is subject to diurnal variation, with highest levels measured early in the morning and a gradual decline seen throughout the remainder of the day. Normal levels of cortisol in serum are about 3-22 μg/dL. Cortisol may also be measured in other biological samples, including human urine (exhibiting a normal level of about 20-90 μg/day) and saliva. Corticosteroids can be modified by endogenous enzymes in the body to form several metabolites that are also detectable in biological samples.

Cortisol-related pathologies can result from both cortisol deficiency and excess, and include Addison's disease, characterized by hypotension, metabolic disturbances, deficient neuromuscular function, decreased resistance to stress, and hyperpigmentation of the skin; secondary adrenal insufficiency; adrenal virilism; and Cushing's syndrome, characterized by truncal obesity, muscle wasting, atrophic skin, hypertension, glucose intolerance, decreased resistance to infection, and psychiatric disturbances. Additionally, because of the anti-inflammatory properties of corticosteroids, they are often used as therapeutics; however, their prolonged use can be related to severe side effects such as muscle wasting and bone resorption.

Because of their clinical importance, numerous assays have been developed to determine levels of one or more corticosteroids in biological samples, but these assays tend to be both time intensive and complicated. The most common methods utilized in clinical laboratories are immunoassays, although HPLC and electrophoretic methods have also been described. Such assays often require very specific antibodies and/or complicated separation methods in order to accurately represent the corticosteroid concentration and avoid undesirable crossreactivity between steroid hormones. For example, cortisol assays in serum can be complicated by steroid binding proteins that sequester cortisol, requiring the use of extraction procedures, and by the detection of other steroids. See, e.g., Rao et al., J. Chromatogr. B. Biomed. Sci. Appl. 25:123-8 (1999); Lee and Goeger, Clin. Biochem. 3:229-33 (1998); Turpeinen et al., Clin. Chem. 43:1386-91 (1997). Additionally, detection of multiple corticosteroids often require that the operator perform multiple assays on a given patient sample.

More recently, mass spectrometry, in combination with off-line extraction and/or chromatographic preparation of samples prior to injection into the mass spectrometer, \ has been applied to the measurement of corticosteroids generally, and cortisol in particular. Such assays are disclosed in, e.g., Nassar et al., J. Chromatogr. Sci. 39:59-64 (2001); Antignac et al, Rapid Comm. Mass Spectrom. 14:33-9 (2000); Gaillard et al., Forensic Sci. Intl. 107:361-79 (2000); Tang et al., J. Chromatogr. B. 742:303-13 (2000); and Dodds et al., J. Ster. Biochem. Mol. Biol. 62:337-43 (1997).

Assays for Bile Acids

Bile acids are powerful emulsifying agents produced in the liver via the metabolism of cholesterol. Bile acids are conjugated in the liver, e.g., with glycine or taurine, to form bile salts. Bile components circulate between the liver and the small intestine to facilitate the absorption of lipids, fatty acids, and lipid-soluble vitamins. Bile acids are also sometimes key indicators of disease or physiological status. The major bile acids are cholic acid (CA), chenodeoxycholic acid (CDCA), and deoxycholic acid (DCA) and their respective taurine and glycine conjugates.

After virtually complete conjugation, bile acids are excreted in bile; in a fasting human, about 50% passes into the gallbladder, and about 50% flows directly into the distal or common bile duct. Bile remaining in the gallbladder is a concentrated solution of bile acids and sodium. Food entering the duodenum stimulates emptying of the gallbladder into the small intestine, from which bile salts, together with associated lipids, fatty acids, and vitamins, are reabsorbed by active transport in the terminal ileum.

Because bile acid levels are specific and sensitive indicators of hepatobiliary disease, measurements of bile acid concentration in serum can be an important indicator of various pathological states. In many patients with liver diseases, the two-hour postprandial bile acid increase is the only detectable abnormality, and is therefore of particular importance.

In addition, an analysis of individual bile acid levels or their combination may assist in the diagnosis and treatment of diseases such as gallstones (cholelithiasis) and primary biliary cirrhosis. For example, the ratio of trihydroxy bile acid to dihydroxy bile acid in serum differentiates patients with obstructive jaundice from those with hepatocellular injury, and the ratio of cholic acid to chenodeoxycholic acid is useful in diagnosing primary biliary cirrhosis and extrahepatic cholestasis.

Also, since intestinal input is a major determinant of the circulating concentrations of bile acids, the level of bile acids is a useful index of ileal dysfunction in Crohns' disease. Therefore, the detection of not only particular bile acids is of medical importance, but also the ability to detect combinations and ratios of bile acids.

Additionally, bile acids are often monitored in pregnancy, as hormonal changes as a result of pregnancy can alter bile transport and metabolism, resulting in pruritis and cholestasis.

Because of their clinical importance, numerous assays have been developed to determine bile acid levels in biological samples, such as enzymatically-based (linked to, e.g., bioluminescent or chemiluminescent labels) and mass spectrophotometric-based assays. See, e.g., Tomer et al., Biomed. Environ. Mass Spectrom. 13:265-72 (1986); Whiting, Clin. Biochem. 20:317-21 (1987); Eckers et al., Rapid Comm. Mass Spectrom. 4:449-53 (1990); Eckers et al., Biol. Mass Spectrom. 20:731-9 (1991); Maeda et al., J. Biolumin. Chemilumin. 8:241-6 (1993); El-Mir et al., Hepatology 26:527-36 (1997); Lemonde et al., Rapid Comm. Mass Spectrom. 13:1159-64 (1999); Bootsma et al., J. Inher. Metab. Dis. 22:307-10 (1999); and Perwaiz et al., J. Lipid Res. 42:114-9 (2001).

Assays for Lamotrigine

Lamotrigine (lamictal) is an antiepileptic drug used to control some types of seizures. Lamotrigine is effective in reducing the frequency of seizures. Lamotrigine is a derivative of dichlorophenyltriazine, and its chemical name is 6-(2,3-dichlorophenyl)-1,2,4-triazine-3,5-diamine. The molecular formula is C₉H₇Cl₂N₅.

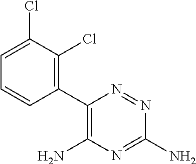 [SMILES: Nc1nnc(-c2cccc(Cl)c2Cl)c(N)n1]

Lamotrigine resembles phenytoin in its pharmacological effects, acting upon sodium channels. It has similar side effects to those of phenytoin (mainly sedation and skin rashes) and no pharmacokinetic anomalies. It is used as adjunctive therapy in the treatment of partial seizure in adults.

The precise mechanism by which lamotrigine exerts its anticonvulsive actions are unknown. In patients administered lamotrigine, the drug is extensively absorbed from the gastrointestinal tract. In the body, metabolism is primarily by glucuronidation. Circulating lamotrigine is about 55% bound to protein at a therapeutic level of 1-10 μg/mL. The half-life of lamotrigine is about 6.4-30.4 hours (single dose) or 7.5-23.1 hours (multiple doses), and phenytoin, carbamazepine, phenobarbital, and/or primidone can reduce this half-life.

Mass spectrometry, HPLC, and capillary electrophoresis methods have been applied to the measurement of lamotrigine in clinical specimens. Such assays are disclosed in, e.g., Dasgupta and Hart, Eur. J. Clin. Chem. 35:755-9 (1997); Castel-Branco et al., J. Chromatogr. B 755:119-27 (2001); Thormann et al., J. Chromatogr. A 924:429-37 (2001).

The contents of each reference cited in the foregoing discussion is hereby incorporated by reference in its entirety, including all tables, figures, and claims.

### SUMMARY OF THE INVENTION

The present invention provides methods and compositions for determining the presence or amount of an analyte of interest, and/or one or more metabolites or precursors of the analyte of interest, in aqueous samples. Preferably, the methods include one or more purification steps that rely on High Turbulence Liquid Chromatography. By permitting the automation of sample preparation and analysis, the methods described herein can reduce costs of assays and the potential for operator error. The methods can permit such assays to be run in a high-throughput fashion, thus providing a rapid, cost effective platform that is particularly useful in a clinical laboratory setting.

In a first aspect, the present invention relates to methods for determining the presence or amount of one or more analytes of interest, and/or one or more metabolites or precursors thereof, in a test sample, comprising ionizing all or a portion of the selected analyte present in the sample to produce one or more analyte ions that are detectable in a mass spectrometer, and detecting the ion(s) so produced. The presence or amount of one or more analyte ions can be related to the presence or amount of the analyte in the original test sample. The presence or amount of analyte ion may also be related to the presence or amount of analyte in the test sample by comparison to a reference analyte sample.

These methods may also comprise ionizing all or a portion of the selected analyte present in the sample to produce one or more analyte ions, isolating the analyte ions by mass spectrometry to provide one or more precursor ions, fragmenting the precursor ions to provide one or more daughter ions that are detectable in a mass spectrometer, and detecting the ion(s) so produced. The presence or amount of the analyte ion(s) can be related to the presence or amount of the analyte in the original test sample. Such methods are known in the art as “tandem mass spectrometry.”

In one preferred embodiment, the analyte is sirolimus, the presence or amount of which is determined in said sample. In preferred embodiments, the sirolimus ion detectable in a mass spectrometer has a mass/charge ratio (m/z) of about 931.70 or about 864.76. In particularly preferred embodiments, a precursor ion of sirolimus has an m/z of about 931.70, and a fragment ion has an m/z of about 864.76. Mass spectrometry instruments can vary slightly in determining the mass of a given analyte. Thus, the term “about” in the context of mass of an ion or the m/z of an ion refers to +/−0.5 atomic mass units.

In a second preferred embodiment, the analyte is a corticosteroid. The present invention provides methods and compositions for determining the presence or amount of corticosteroids in aqueous samples. The methods described herein permit the assay of a plurality of corticosteroid analytes, such as cortisol, 18-hydroxy cortisol, 6-β-hydroxy cortisol, and cortisone, in a sample in a single assay procedure, while advantageously eliminating background signals from closely related steroid hormones.

In this embodiment, methods for determining the presence or amount of cortisol in a test sample comprise ionizing all or a portion of the cortisol present in the sample to produce one or more cortisol ions that are detectable in a mass spectrometer operating in negative ion mode, and detecting the ion(s) so produced.

In preferred methods, cortisol is one of a plurality of corticosteroids, the presence or amount of which are determined in said sample. In these embodiments, all or a portion of each corticosteroid of interest present in the sample is ionized to produce a plurality of ions detectable in a mass spectrometer operating in negative ion mode, and one or more ions produced from each corticosteroid of interest are detected by mass spectrometry. Preferred corticosteroids to be determined in such an assay are a plurality of analytes selected from amongst cortisol, 18-hydroxy cortisol, 6-β-hydroxy cortisol, 20-α-dihydrocortisone, 20-β-dihydrocortisone, 20-α-dihydrocortisol, 20-β-dihydrocortisol, and cortisone.

In preferred methods, a corticosteroid is cortisol, and the ions detectable in a mass spectrometer in positive ion mode include a precursor ion with a mass/charge ratio (m/z) of 363.1 and a fragment ion with an m/z of 121.247. In another method, cortisol ions detectable in negative ion mode include a precursor ion with an m/z of 361, and a fragment ion with an m/z of 331.

In a third preferred embodiment, the analyte is a bile acid. The present invention provides methods and compositions for determining the presence and/or amount of bile acids in a test sample by ionizing a test sample comprising one or more bile acids to produce one or more ions of bile acids detectable by mass spectrometry, and detecting the presence or amount of the ion(s) by mass spectrometry. The methods preferably include detecting bile acid ions in both positive and negative ion mode during a single assay. By detecting both positive and negative ions generated from bile acids in a sample, the present methods can permit the assay of a plurality of bile acids in a single assay run. Thus, in these bile acid assays, the present invention contemplates switching a mass spectrometer from negative to positive ion mode (or vice versa) during the analysis of test samples.

These methods may also comprise ionizing all or a portion of the one or more bile acids present in the sample to produce one or more bile acid ions, isolating the bile acid ion(s) by mass spectrometry to provide one or more precursor ions, fragmenting the precursor ions to provide one or more daughter ions that are detectable in a mass spectrometer, and detecting the ion(s) so produced in both positive and negative ion mode during a single assay.

In certain preferred methods, a plurality of different bile acids are detected in a single assay run. In particularly preferred embodiments, the plurality of bile acids is 2, 3, 4, 5, 6, 7, 8, 9, or more different bile acids. In particularly preferred embodiments, the plurality of bile acids is selected from the group consisting of cholic acid, taurocholic acid, glycocholic acid, chenodeoxycholic acid, taurochenodeoxycholic acid, glycochenodeoxycholic acid, deoxycholic acid, taurodeoxycholic acid, glycodeoxycholic acid, ursodeoxycholic acid, glycoursodeoxycholic acid, taurocoursodeoxycholic acid, lithocholic acid, glycolithocholic acid, and taurolithocholic acid.

The mass spectrometry methods described herein may preferably detect ions selected from those ions having a mass/charge (m/z) of about −407.2 (cholic acid), about −514.2 (taurocholic acid), about −464.4 (glycocholic acid), about +393.2 (chenodeoxycholic acid), about −498.3 (taurochenodeoxycholic acid), about −448.3 (glycochenodeoxycholic acid), about −391.1 (deoxycholic acid), about −498.3 (taurodeoxycholic acid), about −448.3 (glycodeoxycholic acid), about −289.3 (cholic acid), about −80.1 (taurocholic acid), about −74.1 (glycocholic acid), about +359.2 (chenodeoxycholic acid), about −80.1 (taurochenodeoxycholic acid), about −74.1 (glycochenodeoxycholic acid), about −354.3 (deoxycholic acid), about −80.1 (taurodeoxycholic acid), and about −74.1 (glycodeoxycholic acid).

The tandem mass spectrometry methods described herein may preferably isolate precursor ions selected from those ions having a mass/charge (m/z) of about 407.2 (cholic acid), about −514.2 (taurocholic acid), about −464.4 (glycocholic acid), about +393.2 (chenodeoxycholic acid), about −498.3 (taurochenodeoxycholic acid), about −448.3 (glycochenodeoxycholic acid), about −391.1 (deoxycholic acid), about −498.3 (taurodeoxycholic acid), about −448.3 (glycodeoxycholic acid); and detect fragment ions selected from those ions having a mass/charge (m/z) of about −289.3 (cholic acid), about −80.1 (taurocholic acid), about −74.1 (glycocholic acid), about +359.2 (chenodeoxycholic acid), about −80.1 (taurochenodeoxycholic acid), about −74.1 (glycochenodeoxycholic acid), about −354.3 (deoxycholic acid), about −80.1 (taurodeoxycholic acid), and about −74.1 (glycodeoxycholic acid).

In a fourth preferred embodiment, the analyte is lamotrigine. The presence or amount of lamotrigine in a sample can be compared to a reference standard. In certain methods, an internal standard is included in the assay as a positive control, and to provide a comparison MS signal from a known amount of a substance. Hydroxyzine may be used as a preferred internal standard, but other internal standards will also function. The following ions are preferred for use for detection and quantitation:

Lamotrigine: 255.9, 210.8 Da

Hydroxyzine (Internal Standard): 375.2, 201.1 Da

These methods may also comprise ionizing all or a portion of the selected analyte present in the sample to produce one or more precursor ions, isolating one or more of the precursor ions by mass spectrometry, fragmenting the precursor ions to provide one or more fragment ions that are detectable in a mass spectrometer, and detecting the fragment ion(s) so produced. The presence or amount of the analyte ion(s) can be related to the presence or amount of the analyte in the original test sample. The precursor ion may be in the gaseous state and the inert collision gas may be nitrogen, argon, or another suitable collision gas. In these embodiments, the following precursor/fragment ion pairs are preferred:

Lamotrigine: 255.9/210.8 Da

Hydroxyzine (Internal Standard): 375.2/201.1 Da

In certain embodiments, the analyte(s) present in a test sample can be purified prior to ionization. Numerous methods are known in the art to purify analytes, including chromatography, particularly high performance liquid chromatography (HPLC), thin layer chromatography (TLC); electrophoresis, including capillary electrophoresis; extraction methods, including ethyl acetate extraction, and methanol extraction; and affinity separations, including immunoaffinity separations; or any combination of the above. In particularly preferred embodiments, samples are subjected to a precipitation step that leaves the analyte in solution prior to use of subsequent purification steps.

Preferred embodiments utilize high turbulence liquid chromatography (HTLC), alone or in combination with one or more purification methods, to purify the analyte of interest in samples. In particularly preferred embodiments, samples are extracted using an HTLC extraction cartridge which captures the analyte, then eluted and chromatographed on a second HTLC column prior to ionization. Because the steps involved in these two HTLC procedures can be linked in an automated fashion, the requirement for operator involvement during the purification of the analyte can be minimized. This can result in savings of time and costs, and eliminate the opportunity for operator error. In certain embodiments, one or more purification steps are performed on line, and more preferably all of the purification and mass spectroscopy steps may be performed in an on line fashion.

Purification in this context does not refer to removing all materials from the sample other than the analyte(s) of interest. Instead, purification refers to a procedure that enriches the amount of one or more analytes of interest relative to one or more other components of the sample. In preferred embodiments, purification can be used to remove one or more interfering substances, e.g., one or more substances that would interfere with detection of an analyte ion by mass spectrometry.

In various embodiments, the corticosteroid analyte, and/or its metabolite(s) or precursor(s), present in a test sample can be ionized by any method known to the skilled artisan. These methods include, but are not limited to, electron ionization, chemical ionization, fast atom bombardment, field desorption, photon ionization, electrospray, and inductively coupled plasma. The skilled artisan will understand that the choice of ionization method can be determined based on the analyte to be measured, type of sample, the type of detector, the choice of positive versus negative mode, etc.

Suitable test samples can include any liquid sample that can contain the analyte of interest and/or one or more metabolites or precursors thereof. For example, samples obtained during the manufacture of an analyte can be analyzed to determine the composition and yield of the manufacturing process. In certain embodiments, a sample is a biological sample; that is, a sample obtained from any biological source, such as an animal, a cell culture, an organ culture, etc. Particularly preferred are samples obtained from a human, such as a blood, plasma, serum, hair, muscle, urine, saliva, tear, cerebrospinal fluid, or other tissue sample. Such samples may be obtained, for example, from a patient; that is, a living person presenting themselves in a clinical setting for diagnosis, prognosis, or treatment of a disease or condition. In certain embodiments where the assay detects sirolimus, samples are obtained from a patient receiving sirolimus as an immunosuppressive agent, such as a transplant recipient.

The mass spectrometer typically provides the user with an ion scan; that is, the relative abundance of each m/z over a given range (e.g., 100 to 1000 amu). The results of an analyte assay, that is, a mass spectrum, can be related to the amount of the analyte in the original sample by numerous methods known in the art. For example, given that sampling and analysis parameters are carefully controlled, the relative abundance of a given ion can be compared to a table that converts that relative abundance to an absolute amount of the original molecule. Alternatively, molecular standards can be run with the samples, and a standard curve constructed based on ions generated from those standards. Using such a standard curve, the relative abundance of a given ion can be converted back into an absolute amount of the original molecule. Numerous other methods for relating the presence or amount of an ion to the presence or amount of the original molecule will be well known to those of ordinary skill in the art.

### BRIEF DESCRIPTION OF THE FIGURES

FIG. 1 depicts the general skeleton of sirolimus.

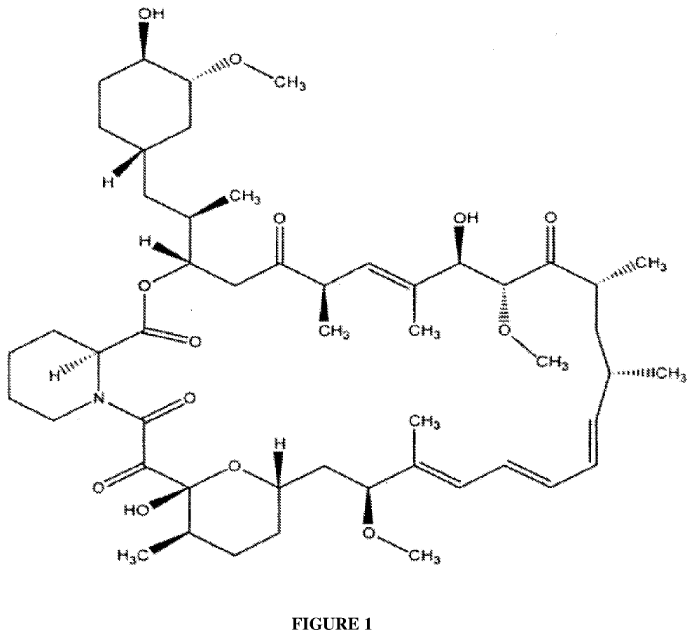

FIG. 2 depicts a product ion scan obtained from a 4.4 ng/mL blood sample containing sirolimus.

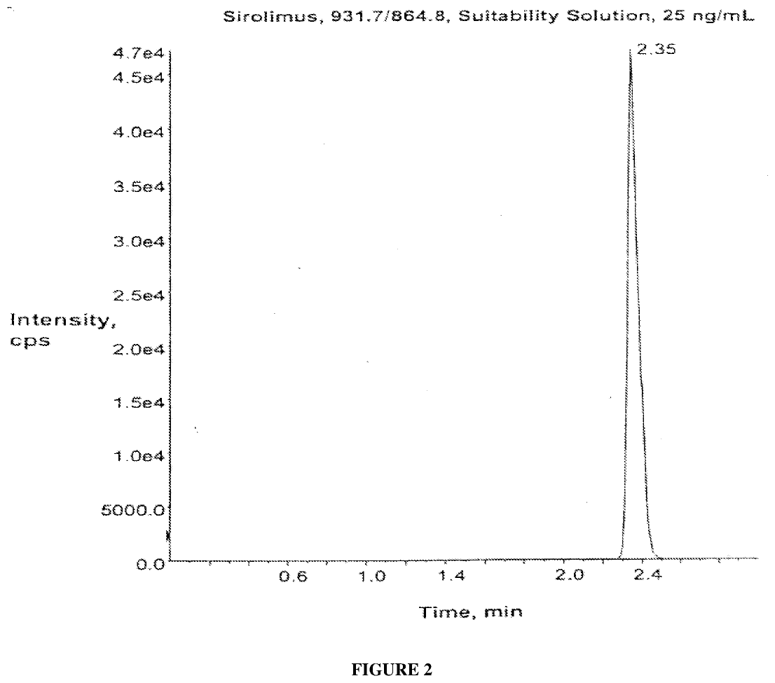

FIG. 3 depicts a diagram of the general pump set-up for HTLC purification of an analyte of interest.

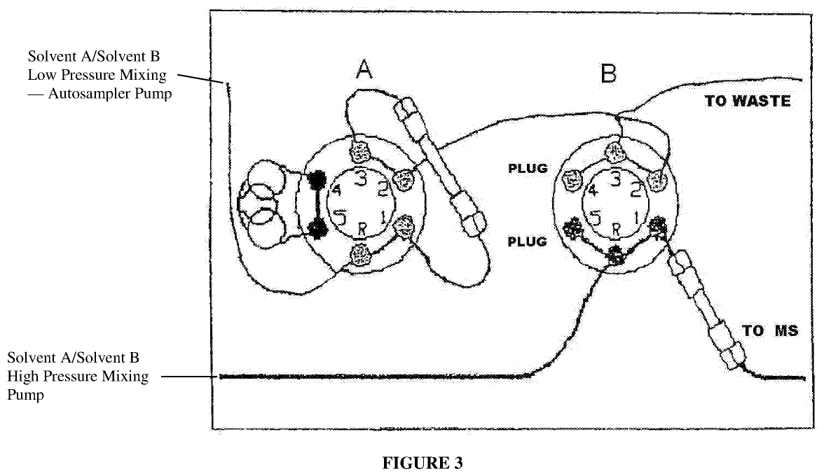

FIG. 4 depicts the pump set up for HTLC purification of sample containing an analyte of interest prior to elution.

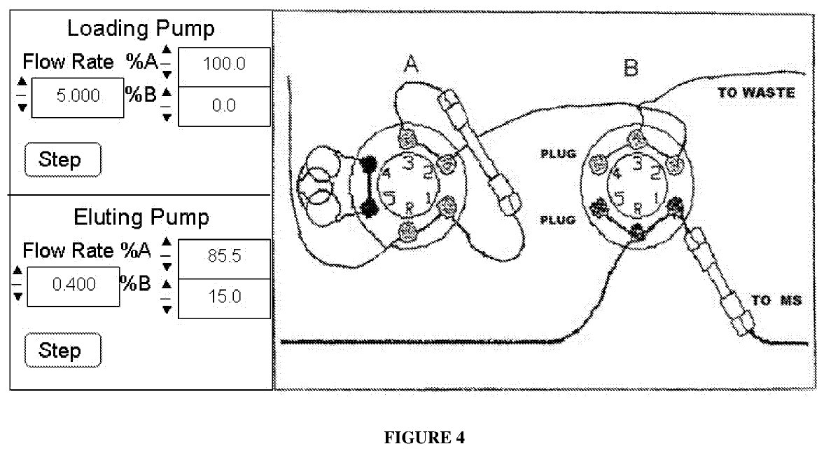

FIG. 5 depicts the pump set up for HTLC reverse elution of an analyte of interest and delivery to mass spectrometer.

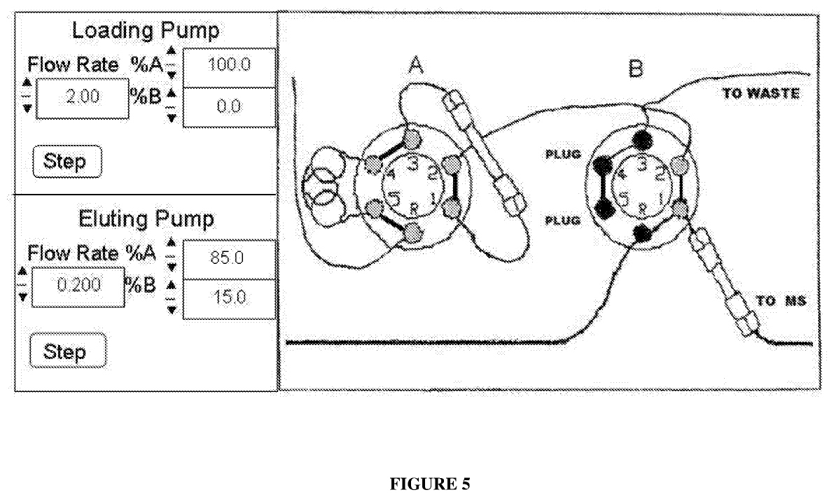

FIG. 6 depicts the pump set up for HTLC column purification.

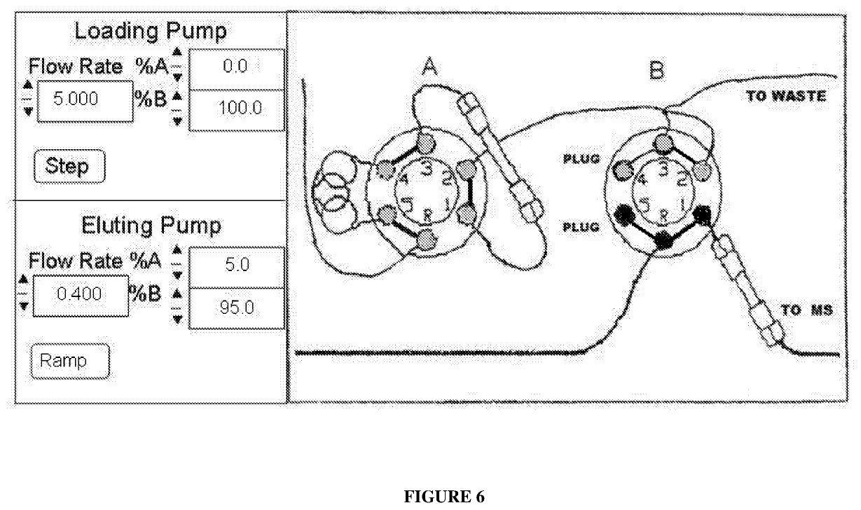

FIG. 7 depicts the general cyclopentanophenanthrene skeleton of corticosteroids, together with several exemplary corticosteroids.

FIG. 8 depicts a product ion scan obtained from a 50 ng/mL urinary cortisol sample run in positive mode, comparing the cortisol signal to that obtained from dexamethasone, and prednisolone.

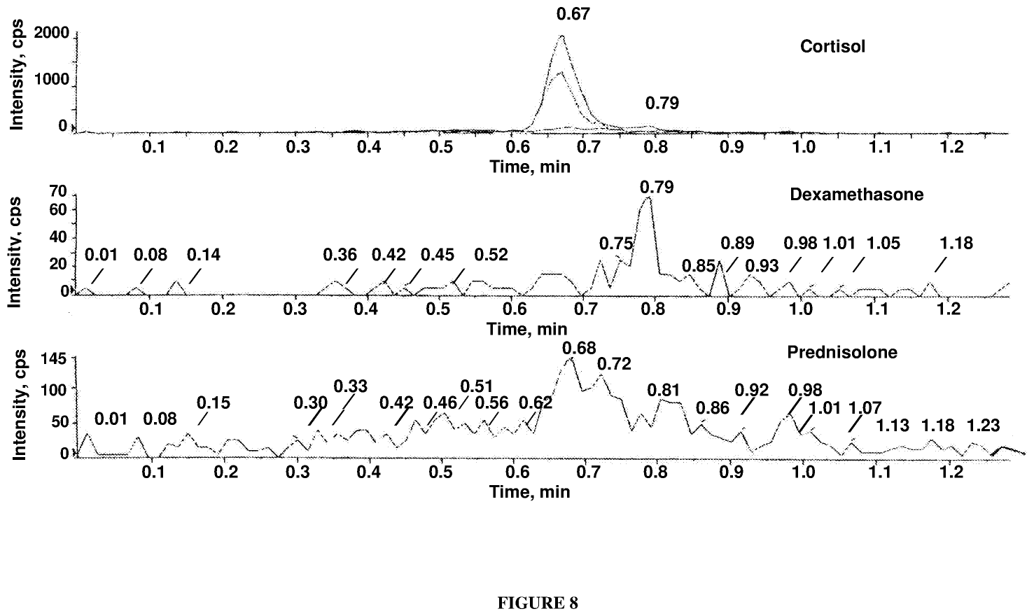

FIG. 9 depicts a product ion scan obtained from a 50 ng/mL urinary cortisol sample run in negative mode.

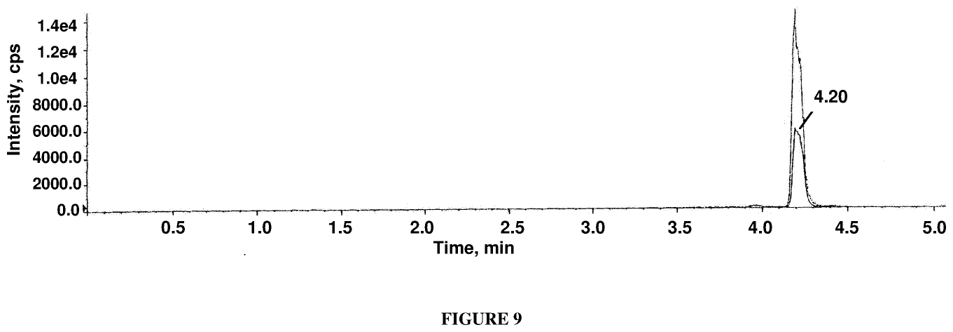

FIG. 10 depicts the general skeleton of various bile acids.

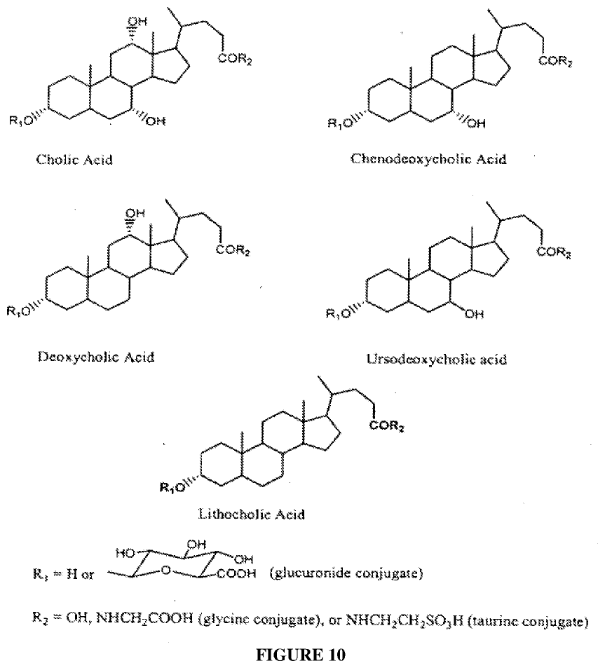

FIG. 11 depicts a product ion scan obtained from a blood sample containing various bile acids in positive mode.

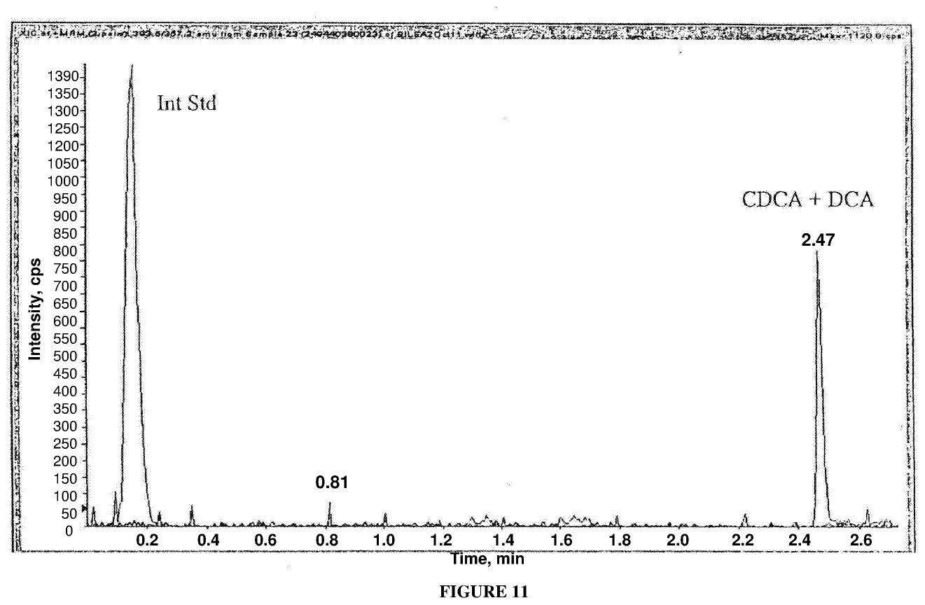

FIG. 12 depicts a product ion scan obtained from a blood sample containing various bile acids in negative mode.

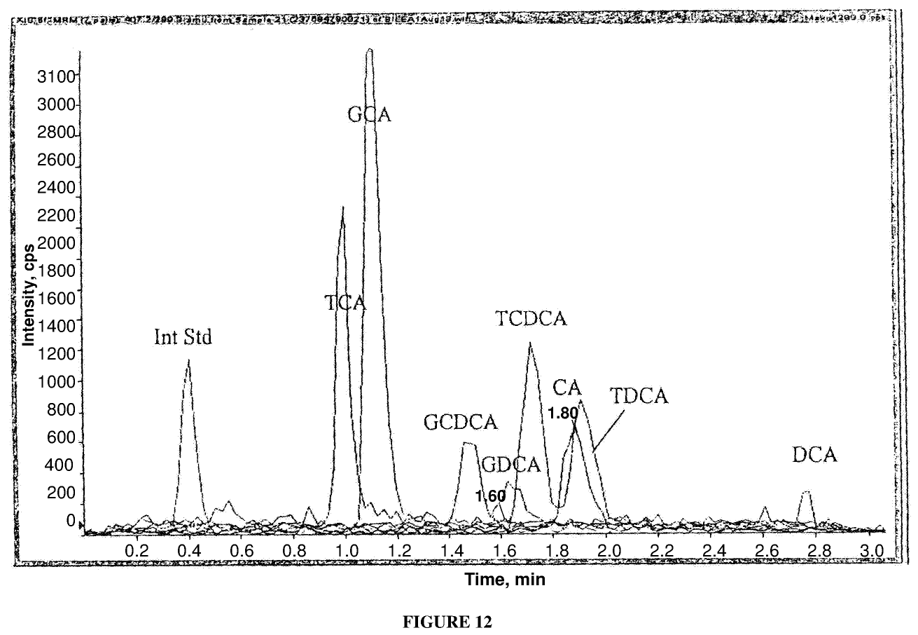

FIG. 13 depicts a product ion scan obtained from a 10 mcg/mL sample containing lamotrigine.

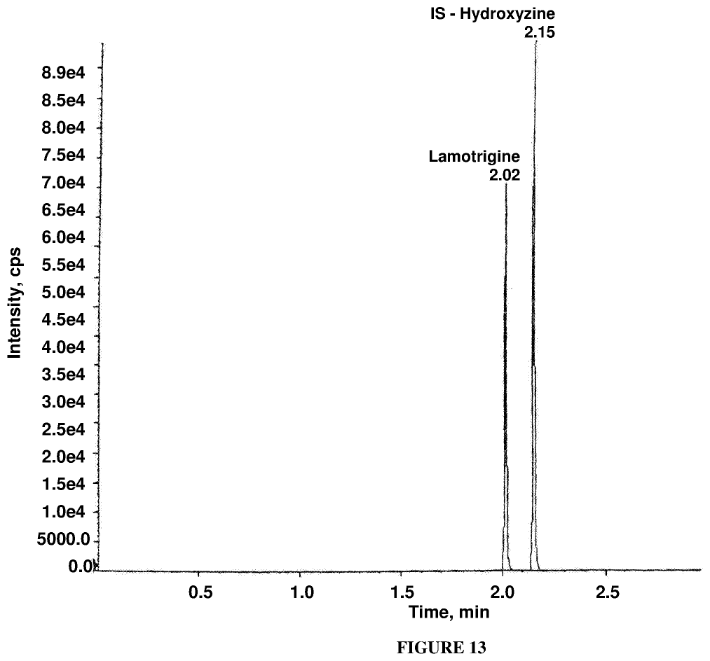

### DETAILED DESCRIPTION OF THE PREFERRED EMBODIMENTS

The present invention describes methods and compositions that can provide the ability to unambiguously detect an analyte of interest, and/or its metabolite(s) or precursor(s), and/or combinations thereof (e.g., multiple bile acids) present in a test sample. By combining mass spectrometry with a multiplexed chromatography system to perform the initial purification of the selected analytes, the present invention can provide a high-throughput assay system particularly well suited to the large clinical laboratory.

Mass Spectrometry

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 2:264-76 (1999); and Merchant and Weinberger, Electrophoresis 21:1164-67 (2000), each of which is hereby incorporated by reference in its entirety, including all tables, figures, and claims.

For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

Moreover, one can often enhance the resolution of the MS technique by employing “tandem mass spectrometry,” or “MS/MS.” In this technique, a first, or parent, ion generated from a molecule of interest can be filtered in an MS instrument, and these parent ions subsequently fragmented to yield one or more second, or daughter, ions that are then analyzed in a second MS procedure. By careful selection of parent ions, only ions produced by certain analytes are passed to the fragmentation chamber, where collision with atoms of an inert gas to produce these daughter ions. Because both the parent and daughter ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Ions can be produced using a variety of methods including, but not limited to, electron ionization, chemical ionization, fast atom bombardment, field desorption, photon ionization, electrospray ionization, and inductively coupled plasma.

The term “electron ionization” as used herein refers to methods in which an analyte of interest in a gaseous or vapor phase is interacted with a flow of electrons. Impact of the electrons with the analyte produces analyte ions, which may then be subjected to a mass spectroscopy technique.

The term “chemical ionization” as used herein refers to methods in which a reagent gas (e.g. ammonia) is subjected to electron impact, and analyte ions are formed by the interaction of reagent gas ions and analyte molecules.

The term “fast atom bombardment” as used herein refers to methods in which a beam of high energy atoms (often Xe or Ar) impacts a non-volatile test sample, desorbing and ionizing molecules contained in the sample. Samples are dissolved in a viscous liquid matrix, such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

The term “field desorption” as used herein refers to methods in which a non-volatile test sample is placed on an ionization surface, and an intense electric field is used to generate analyte ions.

The term “electrospray ionization” or ESI as used herein refers to methods in which a solution is passed along a short length of capillary tube, to the end of which is applied a high positive or negative electric potential. Solution reaching the end of the tube, is vaporized (nebulized) into a jet or spray of very small droplets of solution in solvent vapor. This mist of droplets flows through an evaporation chamber which is heated slightly to prevent condensation and to evaporate solvent. As the droplets get smaller the electrical surface charge density increases until such time that the natural repulsion between like charges causes ions as well as neutral molecules to be released.

The term “Atmospheric Pressure Chemical Ionization,” or “APCI,” as used herein refers to methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N2 gas may be used to improve removal of solvent. The gas-phase ionization in APCI can be more effective than ESI for analyzing less-polar species.

The term “inductively coupled plasma” as used herein refers to methods in which a sample is interacted with a partially ionized gas at a sufficiently high temperature to atomize and ionize most elements.

The term “ionization” as used herein refers to the process of generating an analyte ion having a net electrical charge equal to one or more electron units. Negative ions are those ions having a net negative charge of one or more electron units, while positive ions are those ions having a net positive charge of one or more electron units.

The term “operating in negative ion mode” refers to those mass spectrometry methods where negative ions are detected. Similarly, “operating in positive ion mode” refers to those mass spectrometry methods where positive ions are detected.

The term “desorption” as used herein refers to the removal of an analyte from a surface and/or the entry of an analyte into a gaseous phase.

In those embodiments, such as MS/MS, where parent ions are isolated for further fragmentation, collision-induced dissociation, or “CID,” is often used to generate the ion fragments for further detection. In CID, parent ions gain energy through collisions with an inert gas, and subsequently fragment by a process referred to as “unimolecular decomposition.” Sufficient energy must be deposited in the parent ion so that certain bonds within the ion can be broken due to increased vibrational energy.

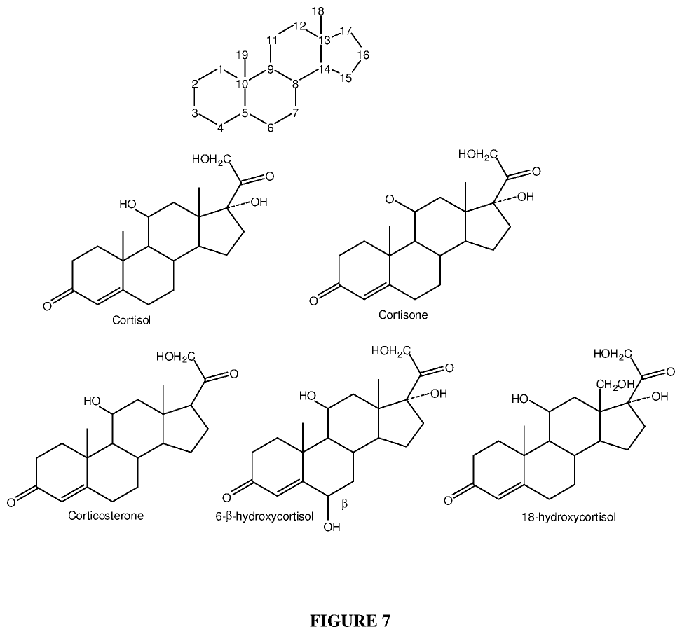

In exemplary embodiments described herein, corticosteroids are analyzed by mass spectrometry. Corticosteroids are a class of steroid hormones and their synthetic relatives constructed on a hydrogenated cyclopentanophenanthrene skeleton. Exemplary corticosteroids are depicted in FIG. 7. While cortisol and cortisone are the primary corticosteroids produced by the adrenal cortex, other metabolites of these parent molecules are often seen in patient samples, including 20-α-dihydrocortisone, 20-β-dihydrocortisone, 20-α-dihydrocortisol, and 20-β-dihydrocortisol. Additionally, closely related steroid hormones, such as prednisolone, estrogen, testosterone, prednisone, etc., may also be present in such samples in significant amounts.

Methods that can distinguish one or more of these corticosteroids in such a background of very closely related molecules generally require extensive purification steps and/or multiple assay runs in order to isolate individual steroid molecules. Because of its high resolution and sensitivity, mass spectrometry can offer the ability to qualitatively or quantitatively measure both parent compounds and metabolites. The methods developed to date, however, can still suffer from interference by closely related steroid molecules.

The present invention describes methods and compositions that can provide the ability to unambiguously detect one or more corticosteroids in a test sample. By careful selection of negative mode ions for detection and/or isolation and fragmentation, assays have been developed that can identify a plurality of corticosteroids simultaneously. Moreover, by combining mass spectrometry with a multiplexed chromatography system to perform the initial purification of the selected analytes, the present invention can provide a high-throughput corticosteroid assay system particularly well suited to the large clinical laboratory.

In other exemplary embodiments, the present invention also describes methods and compositions that can provide the ability to unambiguously detect bile acids, and preferably combinations of bile acids, and/or their metabolite(s), present in a test sample. By combining unique methods of mass spectrometry with a multiplexed chromatography system to perform the initial purification of the selected analytes, the present invention can provide a high-throughput assay system for detecting the presence and amount of bile acids, and is particularly well suited to the large clinical laboratory. The present invention also allows for the separation and detection of combinations of bile acids that have not heretofore been detectable without multiple assay procedures. For example, using the methods of the present invention, one may successfully separate, detect, and quantify many bile acids in a single assay run. The present methods also enable the separation of the dihydroxy bile acids, which are known for being difficult to separate.

In the present invention, it was also discovered unexpectedly that valuable information could be obtained by switching the mass spectrometer from negative to positive ion mode (or vice versa) during analysis of fragment ions. Preferably, the switch between modes takes place just prior to injection into the mass spectrometer for detection. Thus, in MS/MS detection, each injection isolates a selected precursor ion, fragments that precursor ion, and detects a single bile acid. This process is repeated, switching the ion mode as necessary for each detection. It had previously been believed that switching from one mode to the other during the mass spectrometry analysis results in unreliable, inaccurate data. But in the present case of the analysis of bile acids, switching ion modes was found to yield useful and reliable data and result in a substantial savings in time and resources.

Sample Preparation for Mass Spectrometry

Numerous methods have been described to purify analytes of interest from samples prior to assay. For example, high performance liquid chromatography (HPLC) has been used for sample clean-up. See, e.g., Taylor et al., Therapeutic Drug Monitoring 22:608-12 (2000) (manual precipitation of blood samples, followed by manual C18 solid phase extraction, injection into an HPLC for chromatography on a C18 analytical column, and MS/MS analysis); Salm et al., Clin. Therapeutics 22 Supl. B:B71-B85 (2000) (manual precipitation of blood samples, followed by manual C18 solid phase extraction, injection into an HPLC for chromatography on a C18 analytical column, and MS/MS analysis); and Hallensleben et al., J. Am. Soc. Mass Spectrom. 11:516-25 (2000) (incubation of Sirolimus with microsomes to generate metabolites, followed by off-line HPLC chromatography and manual collection of metabolites for analysis by MS).

Taking corticosteroids as an example, numerous methods have been described to purify corticosteroids from samples prior to assaying for one or more corticosteroids. For example, chromatography, particularly high performance liquid chromatography (HPLC), thin layer chromatography (TLC), with and without an extraction step, have been used for sample clean-up. See, e.g., Tang et al., J. Chromatogr. B. 742:303-13 (2000) (multiple extractions followed by reverse phase HPLC); Antignac et al, Rapid Comm. Mass Spectrom. 14:33-9 (2000) (hydrolysis of corticosteroids, followed by C-18 reverse phase HPLC and liquid/liquid extraction); Dodds et al., Anal. Biochem. 247:342-7 (1997) (C-18 reverse phase HPLC followed by centrifugation); Dodds et al., J. Steroid Biochem. Mol. Biol. 62:337-43 (1997) (C-18 reverse phase HPLC); Gaillard et al., Forensic Sci. Intl. 107:361-79 (2000) (C-18 reverse phase HPLC, hydrolysis, and liquid-liquid extraction); Nassar et al., J. Chromatogr. Sci. 39:59-64 (2001) (C-18 analytical reverse phase HPLC).

Numerous methods have been described to purify bile acids from samples prior to assay. For example, high performance liquid chromatography (HPLC) has been used for sample purification and analysis. See, e.g., Yoshida et al., J. Chromatogr. 431:27-36 (1988).

Purification in this context does not refer to removing all materials from the sample other than the analyte(s) of interest. Instead, purification refers to a procedure that enriches the amount of one or more analytes of interest relative to one or more other components of the sample. In preferred embodiments, purification can be used to remove one or more interfering substances, e.g., one or more substances that would interfere with detection of an analyte ion by mass spectrometry.

Recently, high turbulence liquid chromatography (“HTLC”) has been applied for sample preparation of samples containing two unnamed drugs prior to analysis by mass spectrometry. See, e.g., Zimmer et al., J. Chromatogr. A 854:23-35 (1999); see also, U.S. Pat. Nos. 5,968,367; 5,919,368; 5,795,469; and 5,772,874, each of which is hereby incorporated by reference in its entirety. Traditional HPLC analysis relies on column packings in which laminar flow of the sample through the column is the basis for separation of the analyte of interest from the test sample. The skilled artisan will understand that separation in such columns is a diffusional process. In contrast, it is believed that turbulent flow, such as that provided by HTLC columns and methods, may enhance the rate of mass transfer, improving the separation characteristics provided.

Additionally, the commercial availability of HTLC apparatuses that permit multiplexing of columns and direct integration with MS instruments makes such instruments particularly well suited to high-throughput applications.

Numerous column packings are available for chromatographic separation of samples, and selection of an appropriate separation protocol is an empirical process that depends on the sample characteristics, the analyte of interest, the interfering substances present and their characteristics, etc. For HTLC, polar, ion exchange (both cation and anion), hydrophobic interaction, phenyl, C-2, C-8, and C-18 columns are commercially available. Similar columns are also available for traditional HPLC and low pressure separations. During chromatography, the separation of materials is effected by variables such as choice of eluent (also known as a “mobile phase”), choice of gradient elution and the gradient conditions, temperature, etc.

The particles of a typical column packing material are typically greater than 3 μm in average diameter, more preferably greater than 5 μm in average diameter, and preferably may be about 3-10 μm in diameter. Columns can also contain packing material comprising spherical alumina, titania, carbon, and other materials. The person of ordinary skill will realize that other columns may be utilized successfully by following the selection guidelines provided herein. Such packing material may exhibit an extremely narrow particle size and pore size distribution.

For purification of lamotrigine, it has been discovered that a column packed with a styrene-divinylbenzene cross-linked copolymer produces an advantageous separation. Most preferably, the HTLC may be followed by HPLC on a C18 column with a porous spherical silica. During chromatography, the separation of materials is effected by variables such as choice of eluant (also known as a “mobile phase”), choice of gradient elution and the gradient conditions, temperature, etc.

In certain embodiments, an analyte may be purified by applying a sample to a column under conditions where the analyte of interest is reversibly retained by the column packing material, while one or more other materials are not retained. In these embodiments, a first mobile phase condition can be employed where the analyte of interest is retained by the column, and a second mobile phase condition can subsequently be employed to remove retained material from the column, once the non-retained materials are washed through. The second mobile phase may be phased in gradually, usually under computer control directing the composition of mobile phase over time, or by an immediate change in the mobile phase. The retained materials may also be removed from the column by “backflushing” the column, or reversing the direction of flow of the mobile phase. This may be particularly convenient for material that is retained at the top of the column. Alternatively, an analyte may be purified by applying a sample to a column under mobile phase conditions where the analyte of interest elutes at a differential rate in comparison to one or more other materials. As discussed above, such procedures may enrich the amount of one or more analytes of interest relative to one or more other components of the sample.

The terms “phenyl,” “C-2,” “C-8,” and “C-18” as used herein refer to functional groups present on a column packing material. For example, a phenyl column exposes the material flowing through the column to unsubstituted phenyl groups, while a C-18 column exposes the material flowing through the column to unsubstituted straight or branched chain 18-carbon alkyl groups.

The term “analytical column” as used herein refers to a chromatography column having sufficient chromatographic “plates” to effect a separation of materials in a sample that elute from a column sufficient to allow a determination of the presence or amount of an analyte. Such columns are often distinguished from “extraction columns,” which have the general purpose of separating, or extracting, retained from non-retained materials. Examples of analytical columns are C-18 columns.

Preferred analytical columns in the present invention may be monofunctional with polar and bulky end-capping. The columns may be highly retentive and particularly useful for the separation of non-polar to medium polarity compounds using organic/aqueous mobile phases. Columns sold under the name Advantage Lancer® C18 (Cohesive Technologies, Franklin, Mass.), or Hypersil® (company, city, state) columns or similar columns utilizing porous spherical silica are examples of suitable analytical columns for use in the present invention.

Additionally, workers have described the use of affinity binding for sample purification in mass spectrometry. For example, U.S. Pat. Nos. 6,020,208 and 6,153,389, each of which is hereby incorporated by reference in its entirety, disclose the use of antibodies directed against a particular analyte as a ligand receptor to extract and concentrate the analyte.

In preferred embodiments, one or more of the purification and/or analysis steps can be performed in an “on-line” fashion. The term “on-line” as used herein refers to steps performed without the need for operator intervention. For example, by careful selection of valves and connector plumbing, two or more chromatography columns can be connected as needed such that material is passed from one to the next without the need for any manual steps. In preferred embodiments, the selection of valves and plumbing is controlled by a computer pre-programmed to perform the necessary steps. Most preferably, the chromatography system is also connected in such an on-line fashion to the detector system, e.g., an MS system. Thus, an operator may place a tray of samples in an autosampler, and the remaining operations are performed under computer control, resulting in purification and analysis of all samples selected. The commercial availability of HTLC apparatuses that permit multiplexing of columns and direct integration with HPLC and MS instruments makes such instruments particularly well suited to on-line and high-throughput applications.

In contrast, the term “off-line” as used herein refers to a procedure requiring manual intervention of an operator. Thus, if samples are subjected to precipitation, and the supernatants are then manually loaded into an autosampler, the precipitation and loading steps are off-line from the subsequent steps.

### EXAMPLES

The following examples serve to illustrate the present invention. These examples are in no way intended to limit the scope of the invention.

### Example 1. Determination of Sirolimus by Mass Spectrometry

Sample Collection.

Human whole blood was collected in a sterile container and stored refrigerated or at room temperature until analysis. Samples were stable in non-coagulated samples for up to 7 days refrigerated, or 5 days at room temperature. Steady-state (0.5-1 hour pre-oral sirolimus dosage, 2 weeks following beginning of administration of drug) are preferred samples. A minimum volume of 1 mL was collected for an assay.

Sirolimus Assay Procedure.

Samples (0.2 mL) were precipitated by mixing with 0.4 mL ZnSO₄, 0.4 mL acetone, and 0.05 mL internal standard extraction solution (32-desmethoxyrapamycin in 50% aqueous methanol). Following mixing and collection of the supernatant by centrifugation through a PVDF filter, each sample was placed into a well of a standard 96-well plate (MicroLiter Analytical Supplies, Cat. #07-3000). Samples at this stage should be maintained at 4°-8°.

96-well plates were loaded into a HTS PAL autosampler (LEAP Technologies) for injection into the HTLC apparatus. At this point, no further operator handling of samples is required, as the HTLC may be computer-controlled to perform the subsequent purification and analysis steps in a fully on-line configuration.

15±5 uL of each sample was injected onto a PolarPlus™ (C-18 variety) extraction column (Cohesive Technologies No. 952311) in a Cohesive Technologies model 2300 HTLC system synchronized to an API 3000 LC/MS/MS system. Sirolimus is retained by this column, while various other sample substances are eluted.

Flow through the extraction column is reversed, and the column is flushed with a solvent that elutes Sirolimus from the column. This flush solution is injected into a Metachem® C-18-A analytical column (Ansys Technologies Cat. #2000-050x020) fitted with a Polaris C18-A guard column (Cat, #2000-MG2) and eluted with a solvent gradient. A portion of the eluent from this column, representing the Sirolimus peak, is injected into the MS/MS instrument for analysis. Total run time is approximately 3 minutes.

Chromatography was performed under the following conditions:

For low pressure mixing pump,

### Solvent A: 5 mM Ammonium Acetate

### Solvent B: Methanol

| Time (min) | Solvent B | Solvent A | Flow (mL/min) |
|---|---|---|---|
| 0.00 | 10% | 90% | 5.0 |
| 0.49 | 10% | 90% | 5.0 |
| 0.50 | 65% | 35% | 0.2 |
| 1.49 | 65% | 35% | 0.2 |
| 1.50 | 100% | 0% | 5.0 |
| 2.25 | 100% | 0% | 5.0 |
| 2.26 | 10% | 90% | 5.0 |

For high pressure mixing pump,

### Solvent A: 5 mM Ammonium Acetate

### Solvent B: Methanol

| Time (min) | Solvent B | Solvent A | Flow (mL/min) |
|---|---|---|---|
| 0.00 | 30% | 70% | 0.40 |
| 0.49 | 30% | 70% | 0.40 |
| 0.50 | 30% | 70% | 0.20 |
| 1.00 | 30% | 70% | 0.20 |
| 1.01 | 95% | 5% | 0.20 |
| 1.50 | 95% | 5% | 0.40 |
| 2.00 | 95% | 5% | 0.40 |
| 2.01 | 30% | 70% | 0.40 |

MS/MS parameters (can be modified for optimal result)

| | |
|---|---|
| Mode: | Positive Mode |
| Collision Gas | N₂ |
| Cycle time | 0.420 sec |
| Res Q1 | unit |
| Res Q2 | unit |
| Curtain Gas | 8.0 |
| CAD Gas | 6.0 |
| IS | 5500 |
| Temp | 375 |
| NEB | 9.00 |
| TURBO GAS | 7000 |
| DP | 41 (Sirolimus)/46(Int Std) |
| FP | 250 (Sirolimus)/240(Int Std) |
| EP | −10 (Sirolimus)/−10 (Int Std) |
| CE | 23 (Sirolimus)/21 (Int Std) |

Note:
Int Std = Internal Standard

The following mass transitions were used:

Sirolimus: precursor ion (—[NH₃]⁺) 931.70 m/z; fragment ion 864.76 m/z; 32-Desmethoxyrapamycin: precursor ion: 901.68 m/z; fragment ion 834.67 m/z.

### Example 2. Determination of Corticosteroids by Mass Spectrometry

Sample Collection.

Human urine was collected in a sterile container and stored refrigerated or at room temperature until analysis. Samples were stable for up to 7 days refrigerated, or 2 days at room temperature. In addition, samples may be frozen for up to 5 months if necessary. A minimum volume of 0.5 mL was used for an assay.

Cortisol Assay Procedure Using Positive-Mode MS.

Samples were loaded into a Perkin Elmer series 200 autosampler, together with low, medium, and high concentration controls (urine samples spiked with 10-20 ng/mL, 60-100 ng/mL, and 140-180 ng/mL cortisol). 450 uL of each sample was mixed with 450 uL 1% formic acid and vortexed. Samples (50 μL) were injected onto a TurboFlow™ PolarPlus™ extraction column (Cohesive Technologies No. 952242) in a Cohesive Technologies model 2300 HTLC system synchronized to a Perkin Elmer Sciex API 2000 LC/MS/MS system which used a set of four quadrupoles to select and measure ions. Ions were generated by atmospheric pressure chemical ionization (APCI).

Chromatography was performed under the following conditions:

eluant: 0.1% formic acid in methanol;

5.3 min, flow rate HTLC 5 mL/min, analytical column 0.6 mL/min direct to MSMS.

The following mass transitions are used to measure cortisol in the positive mode:

Precursor ion: 363.1 m/z; fragment ion 121.247 m/z.

The total time necessary for each assay is 5.25 minutes. Limitations to the positive mode assay include the fact that certain cortisol metabolites (e.g., 18-hydroxycortisol) cannot be measured, and that high levels of prednisolone and estrogen interfere in specifically detecting the cortisol fragment ion.

Cortisol Assay Procedure Using Negative-Mode MS.

Samples were loaded into a Perkin Elmer series 200 autosampler, together with low, medium, and high concentration controls (urine samples spiked with 10-20 ng/mL, 60-100 ng/mL, and 140-180 ng/mL cortisol). 100 uL of each sample was mixed with 400 uL 5 mM ammonium acetate and vortexed. Samples were injected onto a TurboFlow™ PolarPlus™ extraction column (Cohesive Technologies No. 952242) in a Cohesive Technologies model 2300 HTLC system synchronized to a Perkin Elmer API 2000 LC/MS/MS system.

Chromatography was performed under the following conditions:

eluant 0.1% ammonium acetate in methanol;
5.25 min, flow rate HTLC 5 mL/min, analytical column 0.6 mL/min direct to MSMS.

The following mass transitions are used to measure cortisol in the negative mode:

Cortisol: 361 m/z; fragment ion 331 m/z.; cortisone, 359.0/329.1; 18-hydroxycortisol, 377.2/317.2; 6-β-hydroxycortisol, 377.1/347.2.

Selected MS/MS Parameters.

| | Positive Mode | Negative Mode |
|---|---|---|
| Dwell time | 200 msec | 200 msec |
| Res Q1 | unit | unit |
| Res Q2 | low | low |
| Curtain Gas | 35 | 35 |
| CAD Gas | 3 | 3 |
| NC | 2 | −2 |
| Temp | 485 | 485 |
| GS1 | 60 | 60 |
| GS2 | 15 | 15 |
| DP | 40 | −21 |
| FP | 350 | −350 |
| EP | −10 | 11.5 |
| CE | 33 | −12 |

By detecting cortisol in negative mode, the assay is free of interference from all substances commonly present in human urine, including prednisolone and estrogen. Additionally, cortisone, 18-hydroxycortisol, and 6-β-hydroxycortisol can be measured simultaneously with cortisol, thus reducing the number of assays that must be performed on each sample in order to determine these molecules. The total time necessary for each assay is 5.25 minutes.

Additionally, the HTLC system can be operated with from 1 to up to 4 columns in parallel. Given that a single assay requires 5.25 minutes to traverse the column, by staggering the start time on each column, a 4-fold multiplexed system can inject four times as many test samples into the MS/MS instrument. Thus, a set of 200 samples may be assayed for four different corticosteroids in 263 minutes in both positive and negative modes, as opposed to 1500 minutes by HPLC. Furthermore, following transfer of samples to the autosampler, no further operator handling of samples is required, as the HTLC may be computer-controlled to perform the subsequent purification and analysis steps in a fully on-line configuration.

### Example 3. Determination of Bile Acids by Mass Spectrometry

Sample Collection.

Human whole blood was collected in containers that do not contain anticoagulant, and permitted to clot. A minimum of 0.5 mL serum was used for each assay. Samples were stable for 7 days at room temperature, 14 days at 2°-8° C., and 1 month at −20° C.

The pH was adjusted to pH 4.0 with a 5 mM ammonium acetate solution adjusted to pH 4.0±0.1 with formic acid. The samples were then loaded onto a Perkin Elmer Series 200 autosampler for analysis using an on-line purification/analysis system.

Bile Acid Assay Procedure.

The bile acid samples were analyzed using a HTLC/MS/MS procedure. The samples were first purified by loading them onto a HTLC extraction column (Cohesive Technologies Polar Plus™, Cat #952242; a C-18 reverse phase packing). Following a wash step, the column was backflushed to elute bound bile acids, which were directly loaded on an analytical column (Metachem Technologies Cat #2000-050x020; a C-18 reverse phase packing). The purified bile acids eluted from the analytical column were detected by MS/MS.

Chromatography was performed under the following conditions:

Extraction column wash solution: 5 mM ammonium acetate, 100%; 1.4 minutes, 5.0 mL/min flow rate.

Extraction column eluant: 60:40% Methanol: 5 mM ammonium acetate; 1.0 minutes, 600 μL/min flow rate.

Analytical column eluant: gradient 60% to 100% methanol; 3.0 min, 600 μL/min flow rate.

Selected MS/MS Parameters.

| | | | | |
|---|---|---|---|---|
| Curtain gas | 30.0 | (negative) | 35.0 | (Positive) |
| Collision gas | 4 | (negative) | 4 | (Positive) |
| Nebulizer current | −2.0 | (negative) | 5.0 | (Positive) |
| Temperature | 485 | (negative) | 485 | (Positive) |
| GS1 | 80.0 | (negative) | 80.0 | (Positive) |
| GS2 | 15.0 | (negative) | 15.0 | (Positive) |

| | | |
|---|---|---|
| Dwell time | 200 msec (negative) | 200 msec (Positive) |

| Analyte | DP | FP | EP | CEP | CE | CXP | MODE |
|---|---|---|---|---|---|---|---|
| cholic acid | −66 | −290 | 10 | −21.07 | −44 | −12.34 | negative |
| taurocholic acid | −101 | −350 | 8.5 | −25.32 | −122 | −7.9 | negative |
| glycocholic acid | −61 | −350 | 10.5 | −23.34 | −64 | −7.77 | negative |
| chenodeoxycholic acid | 11 | 350 | −11 | 30.21 | 17 | 27.81 | positive |
| taurochenodeoxycholic acid | −116 | −340 | 9 | −24.69 | −125 | −7.9 | negative |
| glycochenodeoxycholic acid | −61 | −310 | 9 | −22.70 | −66 | −7.774 | negative |
| deoxycholic acid | −76 | −350 | 10.5 | −20.43 | −40 | 13.72 | negative |
| taurodeoxycholic acid | −116 | −340 | 9 | −24.69 | −125 | −7.9 | negative |
| glycodeoxycholic acid | −61 | −310 | 9 | −22.70 | −66 | −7.774 | negative |

The following mass transitions were used:

| Analyte | precursor ion | fragment ion |
|---|---|---|
| cholic acid | −407.2 | −289.3 |
| taurocholic acid | −514.2 | −80.1 |
| glycocholic acid | −464.4 | −74.1 |
| chenodeoxycholic acid | +393.2 | +359.2 |
| taurochenodeoxycholic acid | −498.3 | −80.1 |
| glycochenodeoxycholic acid | −448.3 | −74.1 |
| deoxycholic acid | −391.1 | −354.3 |
| taurodeoxycholic acid | −498.3 | −80.1 |
| glycodeoxycholic acid | −448.3 | −74.1 |

During the analysis, the mass spectrometer was switched from negative ion mode to positive ion mode. This was found unexpectedly to result in the detection of both negative and positive ions in a single assay. This was surprising because it is known in the art that switching from one mode to another during an assay leads to poor, unreliable, and difficult to decipher results. Surprisingly, in the case of the bile acids of the present invention, they were found to be readily detectable with accuracy and reliability, and therefore to save the cost and effort of performing a second assay. Instead, only detection of ions in a second detection mode need be performed. The quantitation of the level of bile acids was based on the abundance of the final fragment ions.

### Example 4. Determination of Lamotrigine by Mass Spectrometry

Sample Collection.

Human whole blood was collected in a sterile container and permitted to clot at room temperature for 20-30 minutes. Serum was collected by centrifugation and collection of supernatant fluid. Samples were stored frozen, refrigerated, or at room temperature until analysis. Samples were stable for up to 2 weeks frozen, 5 days refrigerated, or 2 days at room temperature. A minimum volume of 0.5 mL was collected for this assay.

Lamotrigine Assay Procedure.

Human serum samples suspected of containing lamotrigine, and internal standard solution were aliquoted into a Captiva® 96 well filter plate, which was placed over a 96 well collection plate. Filtration was performed by applying positive pressure (5-10 psi) for 1-2 minutes. Filtered samples were loaded into an autosampler for injection onto a Cyclone® HTLC extraction column (Cohesive Technologies, Inc., Franklin, Mass.). At this point, no further operator handling or attention was required as the process was in-line and automated. Injected sample volume was 15 μL+/−3 μL. An Advantage Lancer® phenyl analytical column (Analytical Sales and Services, Mahwah, N.J., Cat # ADV5976) was used for analysis. The Lancer® HPLC column further purified the sample and provided greater separation of the lamotrigine and internal standard peaks. The sample was eluted using a fast linear gradient to separate the lamotrigine from the internal standard. The sample with the internal standard was then injected onto a tandem mass spectrometer. The HTLC, HPLC, and MS/MS systems were inline and the analysis was performed in an automated fashion.

Chromatography was performed under the following conditions:

Extraction column: Cyclone™, Cat. #952434, Cohesive Technologies, Franklin, Mass.

Extraction and Chromatography Parameters.

(settings can be modified for optimal results)

| | Start | Sec | Flow | Grad | % B | % A | Tee | Loop | Flow | Grad | % B | % A | Comments |
|---|---|---|---|---|---|---|---|---|---|---|---|---|---|
| 0/1 | 0.00 | 30 | 5.00 | Step | 0 | 100 | — | out | 1.00 | Step | 0 | 100 | Load Sample into First (HTLC) Column |
| 2 | 0.50 | 45 | 0.20 | Step | 0 | 100 | T | in | 1.00 | Step | 0 | 100 | Transfer Sample to Second Column |
| 3 | 1.25 | 20 | 5.00 | Step | 100.0 | 0 | — | out | 1.00 | Ramp | 30.0 | 70 | Elute Sample to Second Column |
| 4 | 1.58 | 20 | 5.00 | Step | 100.0 | 0 | — | in | 1.00 | Ramp | 60.0 | 20 | Elute Sample to Second Column |
| 5 | 1.92 | 30 | 5.00 | Step | 100.0 | 0 | — | out | 1.00 | Ramp | 90.0 | 10 | Elute Sample to Second Column |
| 6 | 2.42 | 20 | 5.00 | Step | 100.0 | 0 | — | in | 1.00 | Step | 90.0 | 10 | Elute Sample to Second Column |
| 7 | 2.75 | 30 | 5.00 | Step | 0 | 100 | — | out | 1.00 | Step | 0 | 100 | Re-equilibrate System |

Selected MS/MS Parameters.

| Mode: | Positive Mode |
|---|---|
| Collision Gas | N₂ |
| Dwell time | 100 msec |
| Res Q1 | unit |
| Res Q2 | unit |
| Curtain Gas | 40.0 |
| CAD Gas | 4.0 |
| Temp | 350 |
| GS1 | 50 |
| GS2 | 20 |
| DP | 60 |
| FP | 360 |
| EP | −10 |
| CE | 35 |
| CEP | 14.92 |
| CXP | 0 |

The following mass transitions were used:

lamotrigine: precursor ion 255.9 m/z; fragment ion 210.8 m/z;

hydroxyzine: precursor ion: 375.2 m/z; fragment ion 201.0 m/z

The reportable range of the assay was about 0.5 to about 25 μg/mL. The following drugs were tested as possible interfering substances, all with negative results: acetaminophen, amikacin, aminoguanidine, amiodarone, amitryptyline, amoxicillin, asprin, benztropine, carbamazepine, chlorpromazine, clomipramine, norclomipramine, clonazepam, clozapine, N-desmethylclozapine, cyclosporine A, cyclosporine C, desipramine, N-desethyl amiodarone, diazepam, 5,5-diphenylhydrantoin, disopyramide, doxepin, nordoxepin, ethambutanol, felbamate, flecainide, flunitrazepam, fluoxetine, norfluoxetine, gabapentin, gemfibrozil, haloperidol, ibuprofen, imipramine, lidocaine, maprotiline, mesoridazine, methylphenidate, mexiletine, nordiazepam, norfluoxetine, nortriptyline, prazepam, propafenone, propranolol, protryptyline, pyrazinamide, rifampicin, risperidone, 9-hydroxyrisperidone, sertraline, N-desmethylsertraline, sulfamethoxazide, thiothixene, thioridazine, trazodone, trimethoprim, and trimipramine.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method for determining the amount of sirolimus in a test sample by tandem mass spectrometry, comprising:
(a) purifying sirolimus from said test sample;
(b) ionizing sirolimus purified from said sample to produce lamotrigine precursor ions detectable by mass spectrometry;
(c) fragmenting said precursor ion into one or more fragment ions detectable by mass spectrometry; and
(d) detecting the amount of one or more of the ions of steps (b) and (c) ions by mass spectrometry, wherein the amount of ions detected is related to the amount of lamotrigine in the test sample.

2. The method of claim 1, wherein the purifying in step (a) comprises subjecting said test sample to high performance liquid chromatography (HPLC).

3. The method of claim 2, wherein said HPLC is conducted with a phenyl analytical column.

4. The method of claim 1, wherein the purifying in step (a) comprises extracting lamotrigine from said test sample with a high turbulence liquid chromatography (HTLC) column.

5. The method of claim 4, wherein said HTLC extraction column is a C-18 extraction column.

6. The method of claim 4, wherein said HTLC extraction column comprises a styrene-divinylbenzene cross-linked copolymer packing material.

7. The method of claim 1, wherein said ionizing of step (b) is conducted in positive ion mode.

8. The method of claim 1, wherein the purified sirolimus is ionized by electrospray ionization.

9. The method of claim 1, wherein said ionizing of step (b) is conducted in positive ion mode.

10. The method of claim 1, wherein the test sample comprises blood, plasma, or serum.

11. The method of claim 1, wherein the test sample is obtained from a patient receiving sirolimus as an immunosuppressive agent.

12. The method of claim 1, wherein said precursor ions comprise a mass to charge ratio (m/z) of about 931.70.

13. The method of claim 1, wherein said fragment ions comprise a mass to charge ratio (m/z) of about 864.76.
